(19) United States
(12) Patent Application Publication
(10) Pub. No.: US 2005/0154265 A1
(43) Pub. Date: Jul. 14, 2005

(54) Intelligent nurse robot

(76) Inventors: Xavier Anguera Miro, Tarragona (ES); Jean-Claude Junqua, Santa Barbara, CA (US)

Correspondence Address: HARNESS, DICKEY & PIERCE, P.L.C., P.O. BOX 828, BLOOMFIELD HILLS, MI, 48303, US

(21) Appl. No.: 10/755,862

(22) Filed: Jan. 12, 2004

(51) Int. Cl. A61B 5/00; G06F 17/60; A61B 5/02

(52) U.S. Cl. 600/300; 600/549; 600/365; 600/500; 705/3

(57) ABSTRACT

A robotic nursing system for use with a patient comprises a nursing robot having at least one patient condition sensor, a transmitter, and a receiver mounted therein. A display device for displays data sensed by the patient condition sensor. The display device includes a receiver in communication with the nursing robot. The nursing robot senses patient physiological conditions using the patient condition sensor and transmits the physiological conditions to the display device using the transmitter. The display device then displays the physiological conditions for review by a user. One or another or both. The nursing robot also transmits the physiological conditions to a patient database for storage.

### FIELD OF THE INVENTION

The present invention relates to providing automated nursing care, and more particularly, to an intelligent nurse robot for providing automated nursing care.

### BACKGROUND OF THE INVENTION

The average age of nurses in hospitals and retirement homes is steadily increasing, however, fewer people become nurses every year. Meanwhile, the average age of the population is also increasing and therefore there is a larger demand for nursing services. This has led to an increasing shortage of trained nurses in health systems in almost every industrialized country. Accordingly, there is a need for a solution to this deficiency of the number of working nurses assisting our elderly population.

One solution is to use a robotic nurse as a remote presence in hospitals and retirement homes. In the past, these robots have been designed to be teleoperated by a qualified person not physically located in the same location as the robot. Essentially, the robot acts as a medium to allow communication between the operator and the patient. This still requires, however, a continuous monitoring presence by the teleoperator as the teleoperator controls the robot and, therefore, many of the same problems still exist (i.e. a shortage of qualified operators). Accordingly, there is a need in the art for an improved solution to the nursing shortage.

### SUMMARY OF THE INVENTION

A robotic nursing system for use with a patient comprises a nursing robot having at least one patient condition sensor, a transmitter, and a receiver mounted therein. A display device for displays data sensed by the patient condition sensor. The display device includes a receiver in communication with the nursing robot. The nursing robot senses patient physiological conditions using the patient condition sensor and transmits the physiological conditions to the display device using the transmitter. The display device then displays the physiological conditions for review by a user.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an exemplary illustration of an intelligent nurse robotic system designed according to the principles of the present invention;

FIG. 2 is a schematic view of the intelligent nurse robotic system of the present invention; and

FIG. 3 is an exemplary decision tree used by the intelligent nurse robotic system of the present invention.

### DETAILED DESCRIPTION OF THE PREFERRED EMBODIMENTS

The following description of the preferred embodiment(s) is merely exemplary in nature and is in no way intended to limit the invention, its application, or uses.

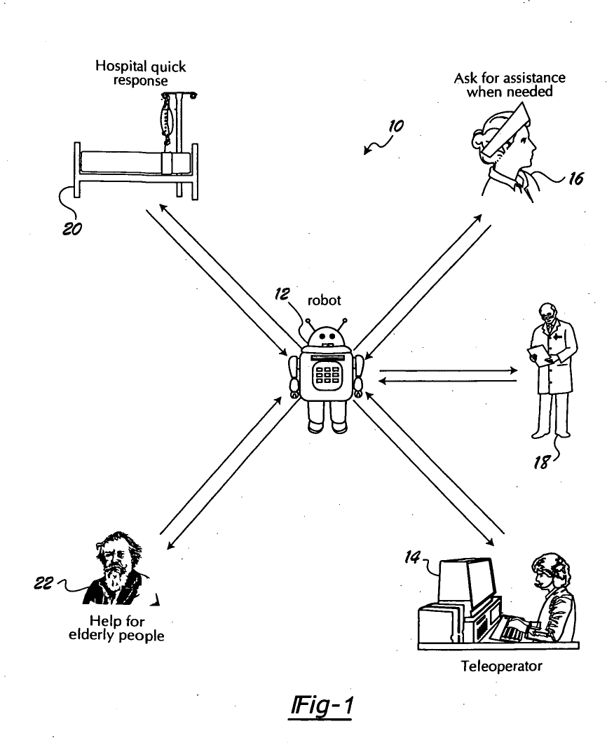

With reference to FIG. 1, an intelligent nurse robotic system 10 is illustrated in an exemplary environment. The intelligent nurse robotic system 10 generally includes an automated device 12, for example a robot, in communication with a teleoperator 14. As will be described below, the robot 12 and the teleoperator 14 work to carry out many of the time consuming tasks required by nursing personnel, such as going from patient to patient in an institution at regular time intervals to measure physiological indicators or responding to non-urgent calls from patients. In this regard, the robot 12 ideally interacts with nurses 16, doctors 18, bedridden patients in hospitals 20 and elderly people 22 in retirement homes.

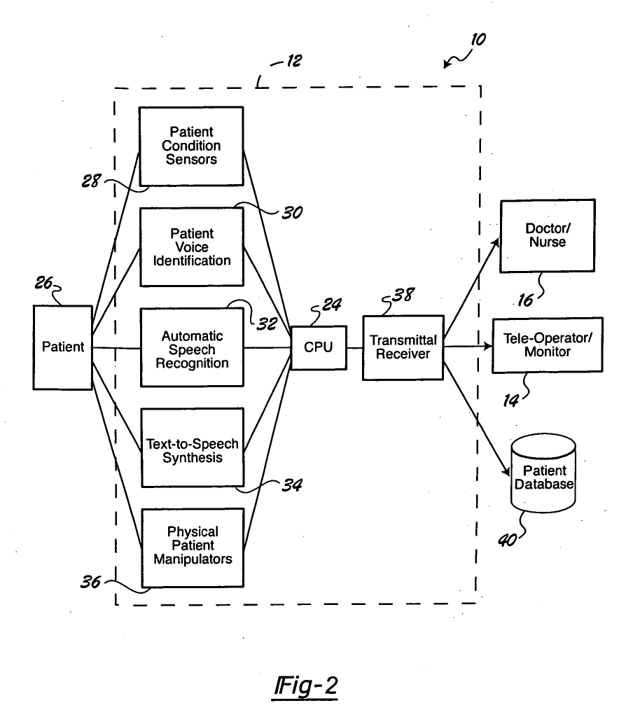

Turning now to FIG. 2, the intelligent nurse robotic system 10 will be described in detail. The robot 12 of the intelligent nurse robotic system 10 generally includes a CPU 24 located therein. The robot 12 further includes a plurality of devices used to interact with a patient 26. These devices include patient condition sensors 28, patient voice identification 30, automatic speech recognition 32, text and speech synthesis 34 and patient manipulators 36. The robot 12 also includes a transmitter/receiver 38 used to communicate with third parties, as will be described below.

The patient condition sensors 28 generally include a plurality of devices used to measure the patient's 26 physiological indicators. For example, these indicators may include blood pressure, sugar in blood, or temperature. It should be appreciated that various other physiological indicators might also be sensed. These sensed indicators are then processed by the CPU 24. The robot 12 then makes an internal decision using an algorithm (built with decision trees, neural networks, or other techniques) to decide whether to do further measurements, administer medicine, or alert a nurse or doctor as to the patient's 26 condition. This independent decision making allows for the robot 12 to be semi-autonomous, as will be described in greater detail below.

The patient voice identification 30 is used to identify the patient 26 using voice identification. For example, this may be accomplished with a password trained in advance that also identifies the voice of the speaker. This assures that patient confidentiality (as required by HIPAA standards) is assured. In an alternate embodiment, the patient condition sensors 28 are also used to identify various biometric factors to be used in an authentication technique (e.g. fingerprints, blood DNA analyses, etc.) either along with or instead of voice identification. Specifically, the patient condition sensors 28 include a biometric identification module used to sense a physiological condition or characteristic of the patient 26 (e.g., such as a camera for facial recognition or an electronic scanning pad for fingerprint identification). The sensed physiological characteristic is then used to identify or recognize a given patient 26. If the patient 26 is new to the intelligent nurse robotic system 10, voice and physiological characteristics may be stored in a patient database 40. The patient database 40 is a data store stored on a server within the hospital or retirement home, though the patient database 40 may also be located within the robot 12 itself. The CPU 24 is in direct communication with the transmitter/receiver 38 and is able to access the patient database 40 to recognize the patient 26 after initial voice and physiological characteristics specific to the patient 26 have been stored therein. The patient database 40 may also include various information specific to a patient 26. For example, such information can include the patient's 26 medical history, the patient's 26 dialogue related preferences (e.g., language and style of interaction), and any other relevant medical information. As will be discussed below, access to the patient database 40 allows the intelligent nurse robotic system 10 to have a great degree of specialization when interacting with a given patient 26.

The automatic speech recognition 32 allows the robot 12 to interact with the patient 26. In this way, the patient 26 may be instructed to use simple word commands in order to communicate with the robot 12. Furthermore, the automatic speech recognition 32 may be relayed through the CPU 24 through the transmitter/receiver 38 and to the teleoperator or monitor such that the teleoperator may hear or see written text of the patient's 26 communications.

The text speech synthesis 34 is used to communicate with the patient 26 using speech. The robot 12 may then inform the patient 26 of any procedures it is performing or any relevant biometric data using a synthesized voice rather than text messages. Moreover, the teleoperator 14 through the transmitter/receiver 38 and the text speech synthesis 44 may directly communicate with the patient 26 through the robot 12. Alternatively, text may be displayed on a screen located on the robot 12 for patient's 26 who are unable to hear or understand audio communication.

The patient manipulators 36 include the actual physical manipulators used to interact with the patient 26 and any services related thereto. These physical manipulators 36 may include arms, trays, sensors or any other interactive device. For example, in order to take the patient's 26 blood pressure, the physical manipulators 36 may include a tray having an automated arm compression portion and sensors that determine the blood pressure of the patient 26.

In retirement homes, the robot 12 may act as a form of entertainment device and companion, used to interact with the patients 26 in various personalized ways. This may include telling stories or adjusting comfort levels for bedridden patients. Entertainment preferences relating to a given patient 26 may be uploaded into the patient database 40.

In the event that the CPU 24 cannot come to a decision or in the event that the CPU 24 determines that further assistance is needed from a human, the robot 12 may communicate directly with a doctor/nurse 16 using the transmitter/receiver 38. The doctor/nurse 16 may receive information from the robot 12 through a PDA, cellular phone or a similar device. The data stream from the patient condition sensors 28 may also be transmitted directly to a device such as a PDA in the doctor/nurse's 16 possession such that the doctor/nurse 16 may look at a patient's 26 physiological measurements in real time or have the robot 12 perform an additional measurement upon request.

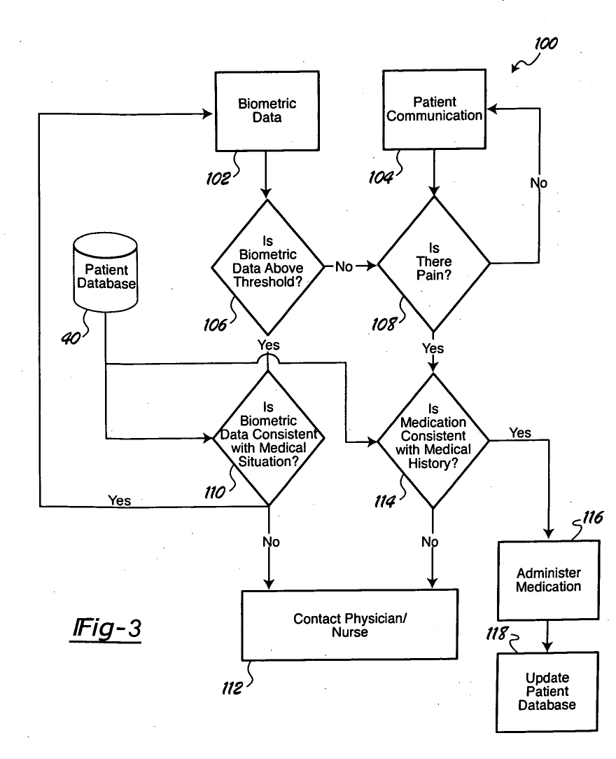

With reference to FIG. 3, the robot 12 uses an expert system to act semi-autonomously. For example, a decision tree is illustrated by reference numeral 100. The decision tree 100 as illustrated is only one of numerous other possible semi-autonomous systems that can be used with the present invention. To begin, the robot 12 senses physiological data of a patient at step 102. Simultaneously, at step 104, the robot 12 is communicating with the patient to determine how the patient is feeling, etc. The robot 12 at step 106 then determines if any of the physiological data is above a given threshold. This threshold is based on medical knowledge and is used to determine the medical condition of the patient. If the physiological data does not exceed the threshold, the robot 12 may go on to determine from communicating with the patent at step 104 to determining if the patient is in pain at step 108.

If, however, the threshold is exceeded at step 106, then the robot 12 then determines if the physiological data is consistent with the patient's medical history by accessing the patent database 40 at step 110. If the physiological data is consistent, then the robot 12 takes no further action. If, however, the physiological data is not consistent, then the robot 12 decides to contact a physician or nurse at step 112.

Similarly, at step 114, the robot 12 can determine if medication used to decrease the patient's pain is consistent with the medical history from the patient database 40. If not consistent, the robot 12 may contact a physician or nurse at step 112. If, however, medication is consistent with the patient's medical history and condition, then the robot 12 may autonomously administer medication to the patient at step 116 and update the patient database with the new medical history at step 118.

Using the above exemplary decision tree, the robot 12 is able to take over many of the tasks currently performed by nurses. Moreover, the physician can tailor the decision tree by altering the thresholds or adding certain medical markers to watch out for (e.g., for a given patient, the physician may want the robot 12 to contact him/her if the patient's heart-rate exceeds a given value, regardless of any other factors). The teleoperator 14 may monitor more than one robot 12 at any given time and take over any given robot 12 as the need arises even if the robot 12 has not decided to contact a physician or nurse, thereby providing a backup to the semi-autonomous robot 12.

The description of the invention is merely exemplary in nature and, thus, variations that do not depart from the gist of the invention are intended to be within the scope of the invention. Such variations are not to be regarded as a departure from the spirit and scope of the invention.

1. A robotic nursing system for use with a patient comprising:
a nursing robot having at least one patient condition sensor, a transmitter, and a receiver mounted therein;
a display device for displaying data sensed by said patient condition sensor, said display device including a receiver in communication with said nursing robot;
wherein said nursing robot senses patient physiological conditions using said patient condition sensor and transmits said physiological conditions to said display device using said transmitter when said nursing robot determines that said physiological conditions must be reviewed; and
said display device displays said physiological conditions for review by a user.

2. The robotic nursing system of claim 1, wherein said patient condition sensor comprises a heart-rate sensor.

3. The robotic nursing system of claim 1, wherein said patient condition sensor comprises a blood-sugar sensor.

4. The robotic nursing system of claim 1, wherein said patient condition sensor comprises a temperature sensor.

5. The robotic nursing system of claim 1, wherein said display device comprises a PDA device.

6. The robotic nursing system of claim 1, wherein said display device comprises a cellular phone device.

7. The robotic nursing system of claim 1, wherein said user is a medically trained individual.

8. A robotic nursing system for use with a patient comprising:
a nursing robot having an at least one patient condition sensor, automatic speech recognition, and text-to-speech synthesis;
wherein said patient condition sensor senses a physiological condition of the patient; and
said nursing robot communicates with the patient using said automatic speech recognition and said text-to-speech synthesis.

9. The robotic nursing system of claim 8, wherein said patient condition sensor comprises a heart-rate sensor.

10. The robotic nursing system of claim 8, wherein said patient condition sensor comprises a blood-sugar sensor.

11. The robotic nursing system of claim 8, wherein said patient condition sensor comprises a temperature sensor.

12. The robotic nursing system of claim 8, wherein said nursing robot further includes voice identification for identifying a voice of the patient to confirm the identity of the patient.

13. The robotic nursing system of claim 12, further comprising a patient database having information relating to the patient, wherein said nursing robot uses said voice identification to identity the patient and accesses said patient database to retrieve information relating to the patient.

14. The robotic nursing system of claim 8, further comprising a patient database for storing information relating to the patient.

15. The robotic nursing system of claim 14, further comprising a transmitter/receiver in communication with said patient database, said transmitter/receiver used to send and receive said information relating to the patient with said patient database.

16. The robotic nursing system of claim 14, further comprising a biometric identification module for identifying the patient, the identity of the patient used to access said information relating to the patient from said patient database.

17. The robotic nursing system of claim 14, wherein said information includes the patient's medical history.

18. The robotic nursing system of claim 14, wherein said information includes the text-to-speech preferences of the patient.

19. The robotic nursing system of claim 8, further comprising a remotely located operator for monitoring said nursing robot.

20. The robotic nursing system of claim 8, wherein said nursing robot further includes a CPU having an internal decision-making tree used to communicate with the patient and to request human intervention when necessary.

21. A robotic nursing system for use with a patient comprising:
a nursing robot having at least one patient condition sensor, a transmitter, and a receiver mounted therein;
a database for storing data sensed by said patient condition sensor, said database including a receiver in communication with said nursing robot;
wherein said nursing robot senses patient physiological conditions using said patient condition sensor and transmits said physiological conditions to said database using said transmitter.

22. A robotic nursing system for use with a patient comprising:
a nursing robot having a biometric identification module and a voice identification module;
wherein said biometric identification module senses a physiological characteristic of the patient;
said voice identification module senses a voice of the patient; and
said nursing robot identifies the patient using said sensed physiological characteristic and said sensed voice.

23. The robotic nursing system of claim 22, wherein said biometric identification module includes a camera and said physiological characteristic includes facial recognition.

24. The robotic nursing system of claim 22, wherein said biometric identification module includes a fingerprint analysis pad and said physiological characteristic includes fingerprint recognition.
